(19) United States
(12) Patent Application Publication
(10) Pub. No.: US 2005/0276722 A1
(43) Pub. Date: Dec. 15, 2005

(54) Apparatus and method for sterilizing an object

(75) Inventor: Mark A. Hilton, Staunton, VA (US)

Correspondence Address: BAKER BOTTS L.L.P., 2001 ROSS AVENUE, SUITE 600, DALLAS, TX, 75201-2980, US

(73) Assignee: MORNINGSTAR SERVICES INC.

(21) Appl. No.: 10/866,581

(22) Filed: Jun. 11, 2004

(51) Int. Cl. A61L 2/00

(52) U.S. Cl. 422/28

(57) ABSTRACT

An apparatus for sterilizing an object includes a chamber that can receive a sterilizing agent. A first screw having a first rod and a first helical thread is coupled to the chamber. The first screw is a right-hand screw that can rotate about the first rod in a first direction. A second screw having a second rod and a second helical thread is coupled to the chamber. The second rod is situated approximately parallel to the first rod. The second screw is a left-hand screw that can rotate about the second rod in a second direction opposite to the first direction. The first helical thread and the second helical thread are positioned to support an object. The first screw and the second screw can continuously move the object in a direction approximately parallel to the first screw through the sterilizing agent to substantially sterilize the object.

### TECHNICAL FIELD

This invention relates generally to the field of sterilization techniques and more specifically to an apparatus and method for sterilizing an object.

### BACKGROUND

Many processes such as food packaging processes require sterilization to remove or kill micro-organisms such as bacteria. Closures for containers, for example, caps for bottles, may be sterilized to provide for a longer shelf life. Some known food packaging techniques sterilize the material for a closure prior to forming the closure. These known techniques, however, cannot effectively be used for certain types of containers such as large bottles. Other known techniques sterilize pre-formed closures. These known techniques, however, have reduced efficiency for certain types of closures such as foil caps. It is generally desirable to have effective and efficient techniques.

### SUMMARY OF THE DISCLOSURE

The present invention provides an apparatus and method for sterilizing an object that substantially reduces or eliminates at least some of the disadvantages and problems associated with previous methods and systems.

In accordance with a particular embodiment of the present invention, an apparatus for sterilizing an object includes a chamber that can receive a sterilizing agent. A first screw having a first rod and a first helical thread is coupled to the chamber. The first screw is a right-hand screw that can rotate about the first rod in a first direction. A second screw having a second rod and a second helical thread is coupled to the chamber. The second rod is situated approximately parallel to the first rod. The second screw is a left-hand screw that can rotate about the second rod in a second direction opposite to the first direction. The first helical thread and the second helical thread are positioned to support an object. The first screw and the second screw can continuously move the object in a direction approximately parallel to the first screw through the sterilizing agent to substantially sterilize the object.

In accordance with another particular embodiment of the present invention, sterilizing an object includes receiving a sterilizing agent into a chamber of an apparatus. The apparatus includes a first screw and a second screw. The first screw has a first rod and a first helical thread and is coupled to the chamber. The first screw is a right-hand screw that can rotate about the first rod in a first direction. The second screw has a second rod and a second helical thread and is coupled to the chamber. The second rod is situated approximately parallel to the first rod. The second screw is a left-hand screw that can rotate about the second rod in a second direction opposite to the first direction. An object is received in the chamber. The first helical thread is positioned with respect to the second helical thread to support the object. The first screw and the second screw are rotated to continuously move the object in a direction approximately parallel to the first screw through the sterilizing agent to substantially sterilize the object.

Particular embodiments of the invention may provide one or more technical advantages. A technical advantage of particular embodiments may be that a pair of screws may be used to convey objects in a substantially continuous motion rather than in a starting and stopping motion. By moving objects in a continuous motion, objects such as foil closures may be more delicately handled.

Another technical advantage of particular embodiments may be that a sterilizing agent for sterilizing objects may be removed from a sterilization chamber after use. The agent may be moved to a region that is separate from the sterilization chamber or other areas where sterilization is occurring to avoid contamination by the used sterilizing agent.

Certain embodiments of the invention may include none, some, or all of the above technical advantages. One or more other technical advantages may be readily apparent to one skilled in the art from the figures, descriptions, and claims included herein.

### BRIEF DESCRIPTION OF THE DRAWINGS

For a more complete understanding of the present invention and its features and advantages, reference is now made to the following description, taken in conjunction with the accompanying drawings, in which:

FIG. 1 is a top view of one embodiment of a system for filling a container that may be used in accordance with the present invention; and

FIGS. 2A through 4 are diagrams illustrating one embodiment of an apparatus for sterilizing an object that may be used with the system filling a container of FIG. 1:

FIG. 2A illustrates a top view of the apparatus that includes a chamber;

FIG. 2B illustrates a top view of the apparatus that shows the interior of the chamber;

FIG. 3 illustrates a side view of the apparatus; and

FIG. 4 illustrates a bottom view of the apparatus.

### DETAILED DESCRIPTION OF THE DRAWINGS

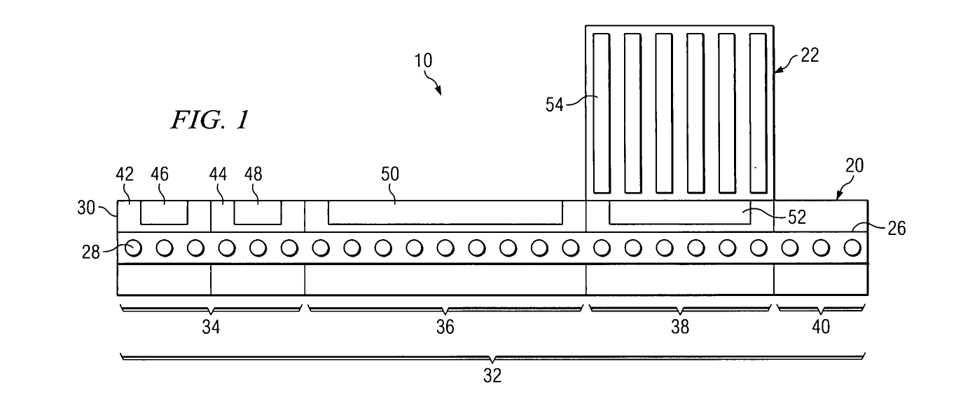

Embodiments of the present invention and its advantages are best understood by referring to FIGS. 1 through 4 of the drawings, like numerals being used for like and corresponding parts of the various drawings.

FIG. 1 is a top view of one embodiment of a system 10 for filling containers. System 10 includes apparatuses for sterilizing closures such as lids before they are applied to the containers. According to the illustrated embodiment, system 10 includes a container filling section 120 coupled to a closure sterilizing section 22. Container filling section 20 sterilizes, fills, and closes containers 28, and closure sterilizing section 22 sterilizes the closures for containers 28 before the closures are applied to containers 28.

According to the illustrated embodiment, a carrier 26 moves containers 28 through container filling section 20. An example container filling section 20 may comprise a container filling system provided by STORK FOOD AND DAIRY SYSTEMS B.V. Container 28 may comprise, for example, a bottle or a jar. Carrier 26 may comprise, for example, a chain type conveyor or a conveyor belt. Container filling section 20 includes a housing 30 that encloses compartments 32. Compartments 32 include a sterilizing zone 34, a filling zone 36, and a closing zone 40. Sterilizing zone 34 operates to substantially sterilize containers 28. Sterilization refers to destroying, neutralizing, or inhibiting the growth of at least some of one or more types of micro-organisms such as bacteria. Sterilization may be used in the preparation of items for purposes such as medical use, food packaging, or other suitable purpose.

Sterilizing zone 34 may include an application zone 42 and a drying zone 44. Application zone 42 includes a sterilization station 46 that applies a sterilizing agent to containers 28. A sterilizing agent comprises an agent, for example, a fluid such as a gas or liquid, that sterilizes. For example, the sterilizing agent may comprise a hydrogen peroxide vapor having a thirty to forty percent, such as a thirty-five percent, concentration of food-grade hydrogen peroxide. The sterilization process may use any number of steps, depending upon the sterilizing agent, the shape of container 28, and the material of container 28. Drying zone 44 includes a drying station 48 that dries containers 28. Drying station 48 may include drying nozzles that blow purified hot air over containers 28.

Filling zone 36 includes a filling station 50 that fills containers 28. Filling station 50 may have filling valves that fill containers with a substance. The substance may comprise a liquid food product such as a dairy product. The filling process may include any number of steps depending upon the substance and container 28. Closing zone 40 includes a closure station 52 that applies a closure to containers 28. A closure may comprise, for example, a cap made of any suitable material such as aluminum foil or plastic that is between fifteen to twenty-five micrometers, such as twenty micrometers thick. The closure, however, may have any suitable thickness, such as between 20 micrometers to five millimeters thick. Closure station 52 receives closures from closure sterilizing section 22. Removal zone 40 is used to remove containers 28 from container filling section 20.

Closure sterilizing section 22 includes apparatuses for sterilizing closures and delivering the closures to closure station 52. Each apparatus may continuously move closures through a sterilizing chamber using two screws. The closures may be sterilized using a sterilizing agent. After use, the sterilizing agent may removed from the sterilizing chamber to a space separate from container filling station 20, thus reducing or avoiding contamination of container filling station 20 with the used sterilizing agent.

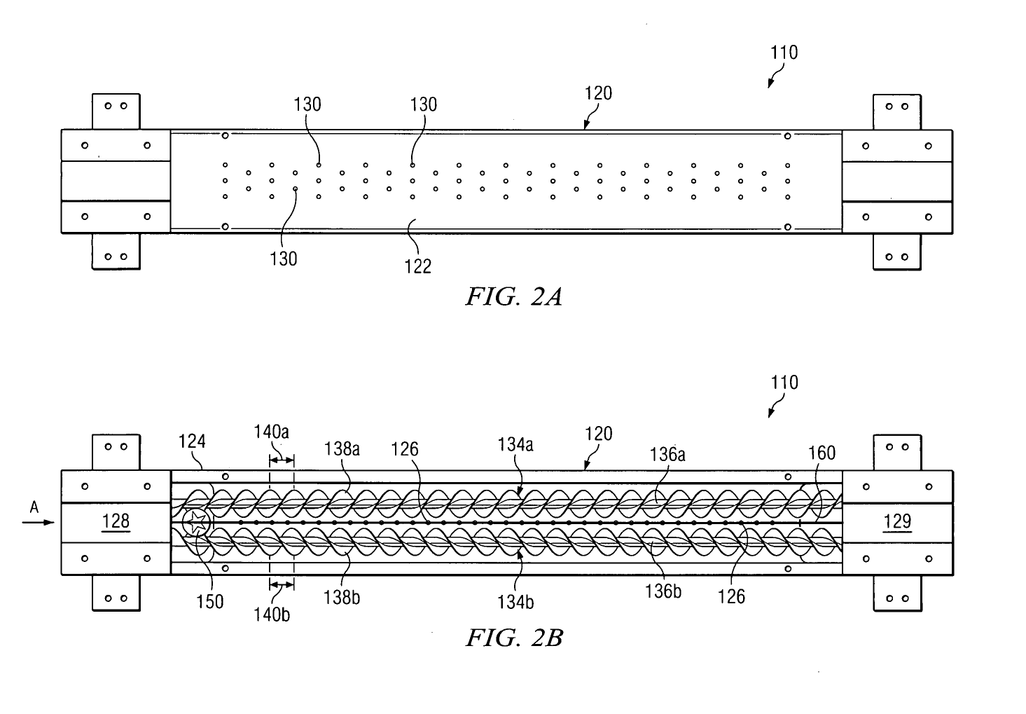

Although closure sterilizing section 22 is shown as having six apparatuses 54, closure sterilizing section 22 may include any suitable number of apparatuses 54. The number of apparatuses 54 may depend upon the number of closures that closure station 52 may accept at one time. An embodiment of an apparatus is described in more detail with reference to FIGS. 2A-4.

Modifications, additions, or omissions may be made to system 10 without departing from the scope of the invention. For example, removal zone 40 may be omitted. Moreover, the operations of system 10 may be performed by more, fewer, or other parts. For example, the operations of application zone 42 and drying zone 44 may be performed by one compartment. Additionally, operations of system 10 may be performed using any suitable logic comprising software, hardware, other logic, or any suitable combination of the preceding. As used in this document, “each” refers to each member of a set or each member of a subset of a set.

FIGS. 2A through 4 are diagrams illustrating one embodiment of an apparatus 110 for sterilizing an object 150. In general, apparatus 110 includes a chamber 120 though which an object 150 may move through during a sterilization process. Apparatus 110 may move object 150 at a substantially constant speed through chamber 120.

FIG. 2A illustrates a top view of apparatus 110 that includes chamber 120. Chamber 120 comprises an enclosure in which object 150 (shown in FIG. 2B) may be sterilized. Chamber 120 may have any suitable size or shape. In general, the width is generally selected to accommodate the diameter of the caps, and the length is generally selected to provide adequate exposure to the sterilizing agent. According to the illustrated embodiment, chamber 120 is an elongated box having dimensions of approximately forty two inches long, three and one-half inches wide, and three and one-half inches high. Chamber 120 may comprise any suitable material such as plastic, metal, other material, or any combination of the preceding. Chamber 120 may have one or more components. According to the illustrated embodiment, chamber 120 has a lid 122 and a body 124 (shown in FIG. 2B).

Object 150 represents an object undergoing a sterilization process. An object may comprise, for example, a closure for a container.

Object 150 may be sterilized by introducing a sterilizing agent into chamber 120. The sterilizing agent may be introduced into chamber 120 using any suitable flow meter operating at any suitable flow rate. For example, eight to twelve milliliters per minute such as approximately 9.75 milliliters per minute of hydrogen peroxide vapor may be applied with a mass flow of three to five standard cubic feet per minute (SCFM) such as approximately 3.75 SCFM. Other suitable flow rates may be used, for example, eight milliliters per minute, six milliliters per minute, or twelve milliliters per minute. The temperature of the hydrogen peroxide vapor may be approximately 250° to 350° Fahrenheit such as approximately 270° Fahrenheit. Objects may be dried at 250° to 300° Fahrenheit such as 270° Fahrenheit.

Lid 122 may have a plurality of inlets 130 that may be used to introduce the sterilizing agent into chamber 120. Lid 122 may have any suitable number of inlets 130 configured in any suitable pattern for distributing a suitable amount of sterilizing agent about object 150 to sterilize object 150. Inlet 130 may have any suitable shape or size. According to the illustrated embodiment, lid 122 has approximately one hundred and thirteen inlets 130, where each inlet is approximately one eighth inch in diameter and approximately one half inch apart from each other.

FIG. 2B illustrates a top view of apparatus 110 with lid 122 removed to expose the interior of chamber 120. Apparatus 110 includes body 124, an entrance 128, an exit 129, screws 134, and a restraint 160 coupled as shown. Body 124 may be configured to receive lid 122 to form an enclosure for the sterilizing agent. Body 124 may comprise any suitable material, for example, metal or plastic such as Teflon. Body 124 may include any suitable number of inlets 126 through which sterilizing agent may be introduced into chamber 120.

Entrance 128 may comprise a ramp used to introduce object 150 into chamber 120, and exit 129 may comprise a ramp used to discharge object 150 from chamber 120. Objects 150 may be placed back to back on the ramp. If objects 150 are substantially circular, screws 134 may capture each object 150 one at a time for transport through chamber 120.

A screw 134 comprises a cylindrical rod 136 having a helical thread 138. Cylindrical rod 136 defines a screw axis about which screw 134 rotates. Pitch refers to the distance 140 measured parallel to the screw axis between corresponding points of adjacent thread forms in the same axial plane and on the same side as the screw axis. According to the illustrated embodiment, cylindrical rods 136a and 136b may have substantially similar lengths and diameters. Pitch 140a may be approximately equal to pitch 140b, and may be selected according to the size of object 150. For example, pitch 140 may be selected to be approximately equal to the size of a dimension such as a diameter or length of object 150.

One screw 134 may be a right-hand screw, and the other screw 134 may be a left-hand screw. A right-hand screw has a right-hand thread that winds in a clockwise and receding direction when viewed axially. A left-hand screw has a left-hand thread that winds in a counterclockwise and receding direction when viewed axially. According to the illustrated embodiment, screw 134b is a right-hand screw, and screw 134a is a left-hand screw.

Screws 134 may be arranged in any suitable configuration to move object 150 in a substantially linear manner. According to the illustrated embodiment, cylindrical rod 136a of screw 134a is substantially parallel to cylindrical rod 136b of screw 134b. The distance between cylindrical rods 136 may be selected such that screws 134 can support object 150 without either screw 134 interfering with the rotation of the other screw 134. For example, the distance may be selected to be approximately equal to the size of a dimension such as a diameter or length of object 150. Helical thread 138a may be situated with respect to helical thread 138b such that helical threads 138 support and convey object 150.

Screw 134a may rotate in a direction opposite from screw 134b in order to move object 150 in one direction. For example, screw 134a may be rotated in a clockwise direction and screw 134b may be rotated in a counterclockwise direction as viewed from direction indicated by arrow A to move object 150 in the direction indicated by arrow A. Similarly, screw 134a may be rotated in a counterclockwise direction and screw 134b may be rotated in a clockwise direction to move object 150a in the direction opposite to the direction indicated by arrow A. Screws 134 may rotate at approximately the same rotational speed. According to one embodiment, screws 134 may be configured and may rotate such that screw 134a is substantially a mirror image of screw 134b.

Restraint 160 may be coupled to chamber 120. Restraint 160 may be used to apply pressure to object 150 in order to hold object 150 in position proximate to helical threads 138.

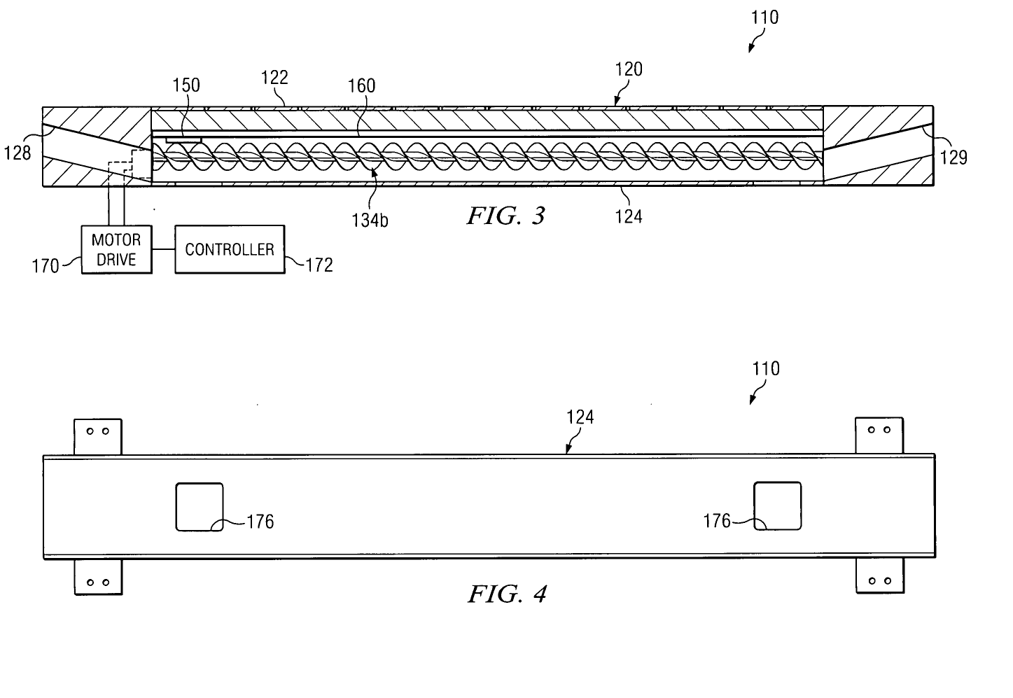

FIG. 3 illustrates a side view of apparatus 110 showing a motor drive 170 and a controller 172. Motor drive 170 is coupled to screws 134 and operates to rotate screws 134. According to one embodiment of the invention, one or more motor drives 170 may rotate a pair of screws 134a and 134b. If a system includes multiple apparatuses 110, each apparatus 110 may have a separate motor drive 170 operable to rotate a pair of screws 134a and 134b. Alternatively, a single motor drive 170 may rotate more than one pair of screws 134a and 134b.

Controller 172 controls the operation of motor drive 170 to rotate screws 134. Controller 172 may include an interface, a processor, and memory integrated or separated according to particular needs. As used in this document, the term “interface” refers to any suitable structure of a device operable to receive input for the device, send output from the device, or both, and may comprise one or more ports. As used in this document, the term “processor” refers to any suitable device operable to execute instructions and manipulate data to perform operations. As used in this document, the term “memory” refers to any structure operable to store and facilitate retrieval of information used by a processor.

Objects 150 may move through chamber 120 at any suitable speed that allows for desired sterilization of objects 150. The speed may be selected in accordance with a predetermined exposure time and the dimensions of chamber 150. The exposure time refers to the duration object 150 should be exposed to a sterilizing agent for desired sterilization. The exposure time may be calculated in accordance with the type of sterilizing agent and the manner that the sterilizing agent is applied to object 150. For example, the exposure time for each cap may be approximately sixty-seven seconds. The speed s of object 150 that should be exposed for exposure time t in chamber 120 of length l may be selected such that s=l/t.

FIG. 4 illustrates a bottom view of apparatus 110. Body 124 has one or more outlets 176 through which the sterilizing agent may be removed from chamber 120 after use. An exhaust air flow may be used to increase the rate of removal. The sterilizing agent may removed from the sterilizing chamber to a space separate from container filling station 20, thus reducing or avoiding contamination of container filling station 20 with the used sterilizing agent.

Alterations or permutations such as modifications, additions, or omissions may be made to apparatus 110 without departing from the scope of the invention. For example, restraint 160 may be omitted. As another example, chamber 120 may comprise more or fewer components.

Particular embodiments of the invention may provide one or more technical advantages. A technical advantage of particular embodiments may be that a pair of screws may be used to convey objects in a substantially continuous motion rather than in a starting and stopping motion. By moving objects in a continuous motion, objects such as foil closures may be more delicately handled.

Another technical advantage of particular embodiments may be that a sterilizing agent for sterilizing objects may be removed from a sterilization chamber after use. The agent may be moved to a region that is separate from the sterilization chamber or other areas where sterilization is occurring to avoid contamination by the used sterilizing agent.

While this disclosure has been described in terms of certain embodiments and generally associated methods, alterations and permutations of the embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

1. An apparatus for sterilizing an object, comprising:
a chamber operable to receive a sterilizing agent;
a first screw coupled to the chamber, the first screw comprising a first rod having a first helical thread, the first screw being a right-hand screw, the first screw operable to rotate about the first rod in a first direction; and
a second screw coupled to the chamber, the second screw comprising a second rod having a second helical thread, the second rod situated approximately parallel to the first rod, the second screw being a left-hand screw, the second screw operable to rotate about the second rod in a second direction opposite to the first direction, the first helical thread positioned with respect to the second helical thread to support an object, the first screw and the second screw operable to continuously move the object in a direction approximately parallel to the first screw through the sterilizing agent to substantially sterilize the object.

2. The apparatus of claim 1, wherein:
the first screw has a first pitch approximately equal to a dimension of the object; and
the second screw has a second pitch approximately equal to the first pitch.

3. The apparatus of claim 1, wherein the chamber further comprises an outlet operable to remove the sterilizing agent from the chamber.

4. The apparatus of claim 1, wherein the chamber further comprises an outlet operable to remove the sterilizing agent from the chamber to a region separate from a container filling section.

5. The apparatus of claim 1, further comprising a motor drive operable to rotate the first screw and rotate the second screw.

6. The apparatus of claim 1, wherein the sterilizing agent comprises hydrogen peroxide.

7. The apparatus of claim 1, wherein the object comprises a pre-formed closure for a container.

8. The apparatus of claim 1, wherein:
the chamber is one of a plurality of chambers, each chamber of the plurality of chambers coupled to a first screw and a second screw; and
the first screw and the second screw of each chamber of the plurality of chambers are rotated by a separate motor drive.

9. A method for sterilizing an object, comprising:
receiving a sterilizing agent into a chamber of an apparatus, the apparatus comprising:
a first screw coupled to the chamber, the first screw comprising a first rod having a first helical thread, the first screw being a right-hand screw, the first screw operable to rotate about the first rod in a first direction; and
a second screw coupled to the chamber, the second screw comprising a second rod having a second helical thread, the second rod situated approximately parallel to the first rod, the second screw being a left-hand screw, the second screw operable to rotate about the second rod in a second direction opposite to the first direction;
receiving an object in the chamber, the first helical thread positioned with respect to the second helical thread to support the object; and
rotating the first screw and the second screw to continuously move the object in a direction approximately parallel to the first screw through the sterilizing agent to substantially sterilize the object.

10. The method of claim 9, wherein:
the first screw has a first pitch approximately equal to a dimension of the object; and
the second screw has a second pitch approximately equal to the first pitch.

11. The method of claim 9, further comprising removing the sterilizing agent from the chamber through an outlet of the chamber.

12. The method of claim 9, further comprising removing the sterilizing agent from the chamber through an outlet of the chamber to a region separate from a container filling section.

13. The method of claim 9, wherein rotating the first screw and the second screw further comprises rotating the first screw and the second screw using a motor drive.

14. The method of claim 9, wherein the sterilizing agent comprises hydrogen peroxide.

15. The method of claim 9, wherein the object comprises a pre-formed closure for a container.

16. The method of claim 9, wherein:
the apparatus is one of a plurality of apparatuses, each apparatus of the plurality of apparatuses comprising a first screw and a second screw; and
wherein rotating the first screw and the second screw further comprises rotating the first screw and the second screw of each apparatus of the plurality of apparatuses using a separate motor drive.

17. A system for sterilizing an object, comprising:
means for receiving a sterilizing agent into a chamber of an apparatus, the apparatus comprising:
a first screw coupled to the chamber, the first screw comprising a first rod having a first helical thread, the first screw being a right-hand screw, the first screw operable to rotate about the first rod in a first direction; and
a second screw coupled to the chamber, the second screw comprising a second rod having a second helical thread, the second rod situated approximately parallel to the first rod, the second screw being a left-hand screw, the second screw operable to rotate about the second rod in a second direction opposite to the first direction;
means for receiving an object into the chamber, the first helical thread positioned with respect to the second helical thread to support the object; and
means for rotating the first screw and the second screw to continuously move the object in a direction approximately parallel to the first screw through the sterilizing agent to substantially sterilize the object.

18. A plurality of apparatuses for sterilizing an object, each apparatus comprising:
a chamber operable to receive a sterilizing agent, the sterilizing agent comprising hydrogen peroxide;
a first screw coupled to the chamber, the first screw comprising a first rod having a first helical thread, the first screw being a right-hand screw, the first screw operable to rotate about the first rod in a first direction;
a second screw coupled to the chamber, the second screw comprising a second rod having a second helical thread, the second rod situated approximately parallel to the first rod, the second screw being a left-hand screw, the second screw operable to rotate about the second rod in a second direction opposite to the first direction, the first helical thread positioned with respect to the second helical thread to support an object, the object comprising a pre-formed closure for a container, the first screw having a first pitch approximately equal to a dimension of the object, the second screw having a second pitch approximately equal to the first pitch, the first screw and the second screw operable to continuously move the object in a direction approximately parallel to the first screw through the sterilizing agent to substantially sterilize the object;
a motor drive operable to rotate the first screw and rotate the second screw; and
an outlet operable to remove the sterilizing agent from the chamber to a region separate from a container filling section.

19. A system for filling a container, comprising:
a container filling section operable to:
accept a plurality of containers;
fill each container of the plurality of containers with a substance; and
close each container of the plurality of containers with a closure of a plurality of closures; and
a closure sterilizing section coupled to the container filling section and operable to sterilize the plurality of closures, the closure sterilizing section comprising one or more apparatuses, each apparatus comprising:
a chamber operable to receive a sterilizing agent;
a first screw coupled to the chamber, the first screw comprising a first rod having a first helical thread, the first screw being a right-hand screw, the first screw operable to rotate about the first rod in a first direction; and
a second screw coupled to the chamber, the second screw comprising a second rod having a second helical thread, the second rod situated approximately parallel to the first rod, the second screw being a left-hand screw, the second screw operable to rotate about the second rod in a second direction opposite to the first direction, the first helical thread positioned with respect to the second helical thread to a closure of the plurality of closures, the first screw and the second screw operable to continuously move the closure in a direction approximately parallel to the first screw through the sterilizing agent to substantially sterilize the closure.

20. The system of claim 19, wherein the container filling section is further operable to sterilize the plurality of containers.
